(19) United States
(12) Patent Application Publication
(10) Pub. No.: US 2025/0253049 A1
(43) Pub. Date: Aug. 7, 2025

(54) INFORMATION PROCESSING APPARATUS, INFORMATION PROCESSING METHOD, AND NON-TRANSITORY STORAGE MEDIUM STORING PROGRAM

(71) Applicant: biomy, Inc., Tokyo (JP)

(72) Inventors: Teppei KONISHI, Tokyo (JP); Hiroyuki Sano, Tokyo (JP); Takuma Kobayashi, Tokyo (JP); Mateusz Andrzej Grynkiewicz, Tokyo (JP); Keita Saito, Tokyo (JP)

(73) Assignee: biomy, Inc., Tokyo (JP)

(21) Appl. No.: 19/189,283

(22) Filed: Apr. 25, 2025

Related U.S. Application Data
(63) Continuation of application No. PCT/JP2023/037387, filed on Oct. 16, 2023.

(30) Foreign Application Priority Data
Oct. 28, 2022 (JP) 2022-173127

(51) Int. Cl. G16H 50/20; G01N 33/574; G16H 20/10

(52) U.S. Cl. CPC G16H 50/20; G01N 33/574; G16H 20/10

(57) ABSTRACT

An information processing apparatus includes a storage that stores a prognosis estimation model, which has been trained to output a prognosis of a patient upon input of input data including a spatial distribution of at least one of a feature value related to a predetermined biomarker or a predetermined protein in a specimen collected from the patient, an acquisition part that acquires a spatial distribution of at least one of a feature value related to a predetermined biomarker and a predetermined protein in a specimen collected from a target patient, and a prognosis estimation part that outputs, as an estimated value of a prognosis of the target patient, information output by inputting input data including the spatial distribution acquired by the acquisition part to the prognosis estimation model.

### CROSS-REFERENCE TO RELATED APPLICATIONS

The present application is a continuation application of International Application number PCT/JP2023/037387, filed on Oct. 16, 2023, which claims priority under 35 U.S.C § 119 (a) to Japanese Patent Application No. 2022-173127, filed on Oct. 28, 2022, contents of which are incorporated herein by reference in their entirety.

### BACKGROUND OF THE INVENTION

The present disclosure relates to an information processing apparatus, an information processing method, and a non-transitory storage medium storing a program.

For example, in the following Non Patent Document, CD4-positive and CD8-positive T cell lymphocytes are known to particularly influence the prognosis of cancer. Studies for performing prognosis prediction using them have been conducted.

Non Patent Document: Rebecca Hoesli, et al.: “Proportion of CD4 and CD8 tumor infiltrating lymphocytes predicts survival in persistent/recurrent laryngeal squamous cell carcinoma”, Oral Oncology, Vol.77, pp 83-89 (February 2018,)

In the related art, information available for prognosis prediction is limited, and thus, improvements in prediction accuracy are also limited.

The present disclosure focuses on this point, and an object thereof is to improve the accuracy of disease prognosis prediction.

### SUMMARY

A first aspect of the present disclosure provides an information processing apparatus including a storage that stores a prognosis estimation model, which has been trained to output a prognosis of a patient upon input of input data including a spatial distribution of at least one of a feature value related to a predetermined biomarker or a predetermined protein in a specimen collected from the patient, an acquisition part that acquires a spatial distribution of at least one of a feature value related to a predetermined biomarker and a predetermined protein in a specimen collected from a target patient, and a prognosis estimation part that outputs, as an estimated value of a prognosis of the target patient, information output by inputting input data including the spatial distribution acquired by the acquisition part to the prognosis estimation model.

A second aspect of the present disclosure provides an information processing method executed by a computer, the method includes the steps of acquiring a spatial distribution of at least one of a feature value related to a predetermined biomarker and a predetermined protein in a specimen collected from a target patient, and outputting, as the estimated value of the prognosis of the target patient, information output by inputting, to a prognosis estimation model stored in a storage, input data including the spatial distribution acquired in the acquiring.

A third aspect of the present disclosure provides a non-transitory storage medium storing a program for causing a computer to realize the steps of acquiring a spatial distribution of at least one of a feature value related to a predetermined biomarker and a predetermined protein in a specimen collected from a target patient, and outputting, as the estimated value of the prognosis of the target patient, information output by inputting, to a prognosis estimation model stored in a storage, input data including the spatial distribution acquired in the acquiring.

### BRIEF DESCRIPTION OF THE DRAWINGS

FIG. 1 is a diagram for explaining an overview of processing in an information processing apparatus 1 according to an embodiment.

FIG. 2 is a block diagram showing a configuration of the information processing apparatus 1.

FIG. 3 shows an example of processing in a distribution generation part 133.

FIG. 4 shows an example of the processing in the distribution generation part 133.

FIG. 5 is a flowchart illustrating processing in the information processing apparatus 1.

FIG. 6 is a diagram for explaining an overview of processing in the information processing apparatus 1 according to a first modified example.

FIG. 7 is a diagram for explaining an overview of processing in the information processing apparatus 1 according to a second modified example.

### DETAILED DESCRIPTION OF THE INVENTION

Hereinafter, the present disclosure will be described through exemplary embodiments of the present disclosure, but the following exemplary embodiments do not limit the disclosure according to the claims, and not all of the combinations of features described in the exemplary embodiments are necessarily essential to the solution means of the disclosure.

### [Overview of Information Processing Apparatus 1]

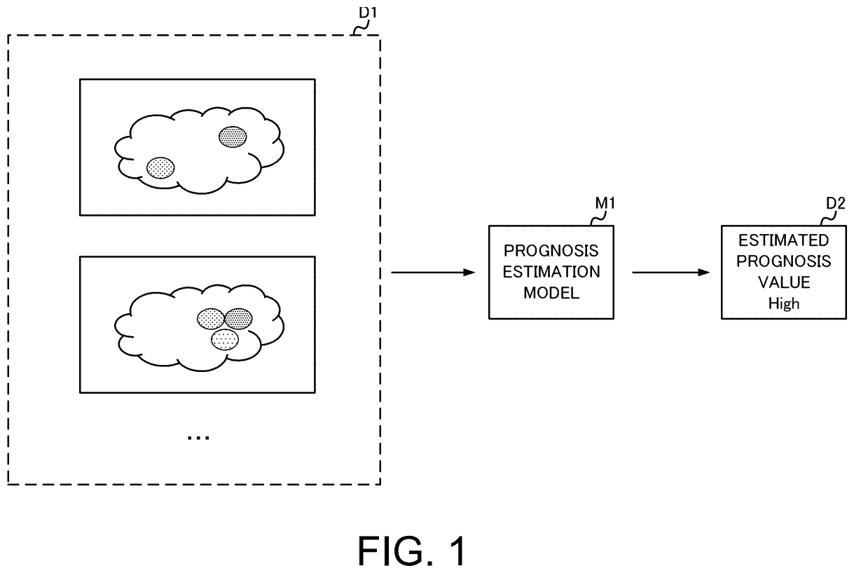

FIG. 1 is a diagram for explaining an overview of processing in an information processing apparatus 1 according to an embodiment. The information processing apparatus 1 is an apparatus for estimating a prognosis of a target patient on the basis of a spatial distribution of a predetermined index in a specimen collected from the patient. The information processing apparatus 1 is a server or a personal computer, for example.

The information processing apparatus 1 inputs input information including a spatial distribution D1 to a prognosis estimation model M1, and outputs information output by the prognosis estimation model M1 as an estimated prognosis value D2. The spatial distribution D1 is information that spatially indicates the extent to which a feature value related to a predetermined biomarker, a tumor tissue, a predetermined type of lymphocyte (protein), or the like are distributed, in image data generated by imaging a specimen collected from a target patient.

The prognosis estimation model M1 is a trained model that has been trained using, as teacher data, spatial distributions of a biomarker, a tumor tissue, a predetermined type of lymphocyte, and the like in a specimen. When the spatial distribution D1 of a target patient is input, the prognosis estimation model M1 outputs the estimated prognosis value D2. The prognosis estimation model M1 may output the estimated prognosis value D2 on the basis of spatial distributions of a plurality of indices. The prognosis estimation model M1 may output the estimated prognosis value D2 on the basis not only of the spatial distribution but also on other input data.

The estimated prognosis value D2 is an estimated value indicating a prognosis of a target patient. As one example, the estimated prognosis value D2 indicates whether or not the probability of the target patient surviving for a predetermined period from a time point when a specimen is obtained is equal to or greater than a predetermined threshold value. FIG. 1 shows an example in which i) the prognosis estimation model M1 outputs, as the estimated prognosis value D2, a value indicating “High” when the probability of the target patient surviving for the predetermined period is equal to or greater than the predetermined threshold value, and ii) the prognosis estimation model M1 outputs, as the estimated prognosis value D2, a value indicating “Low” when the probability is less than the predetermined threshold value. The estimated prognosis value D2 may indicate a period during which the probability of survival of the target patient is estimated to be equal to or greater than the predetermined threshold value, starting from the point at which the specimen was obtained.

The information processing apparatus 1 uses the spatial distribution of the biomarker or the predetermined protein in the specimen for performing the prognosis prediction of the patient, thereby improving the accuracy of disease prognosis prediction as compared with the existing prediction technique.

### [Configuration of Information Processing Apparatus 1]

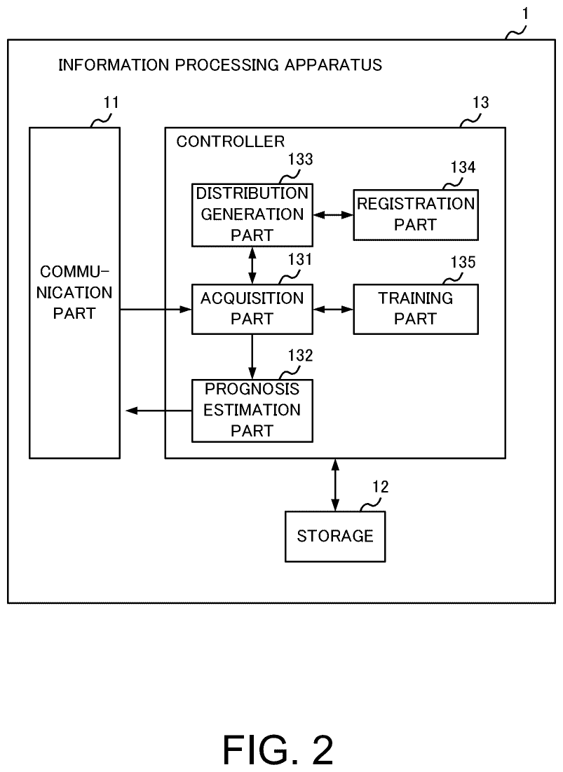

FIG. 2 is a block diagram showing a configuration of the information processing apparatus 1. The information processing apparatus 1 includes a communication part 11, a storage 12, and a controller 13. The controller 13 includes an acquisition part 131, a prognosis estimation part 132, a distribution generation part 133, a registration part 134, and a training part 135.

The communication part 11 is a communication interface for transmitting and receiving data to and from other devices via a network. The storage 12 is a storage medium including a Read Only Memory (ROM), a Random Access Memory (RAM), a Solid State Drive (SSD), a hard disk drive, or the like. The storage 12 stores in advance a program to be executed by the controller 13.

The storage 12 stores the prognosis estimation model M1, which has been trained to output a prognosis of a patient upon input of input data including the spatial distribution of at least one of i) the feature value related to the predetermined biomarker or ii) the predetermined protein in the specimen collected from the patient. The prognosis estimation model M1 is a trained model that has been trained using, as teacher data, i) a spatial distribution of a biomarker or a protein in a specimen collected from a patient and ii) prognosis information of the patient. The prognosis estimation model M1 is trained using, as training data, a spatial distribution of i) a feature value related to a predetermined biomarker and ii) a predetermined protein, corresponding to the spatial distribution acquired by the acquisition part 131.

The controller 13 is a processor such as a Central Processing Unit (CPU), for example. The controller 13 functions as the acquisition part 131, a prognosis estimation part 132, a distribution generation part 133, a registration part 134, and a training part 135 by executing the program stored in the storage 12.

The acquisition part 131 acquires a spatial distribution of at least one of a feature value related to a predetermined biomarker or a predetermined protein in a specimen collected from a target patient. The acquisition part 131 may acquire a spatial distribution of any one of a feature value related to a predetermined biomarker and a predetermined protein, or may acquire spatial distributions of both of them. The acquisition part 131 may acquire the spatial distribution of the feature value related to the predetermined biomarker or the like from an external device which is not shown in figures. As will de described later, the acquisition part 131 may acquire a spatial distribution generated by performing image analysis on the acquired image data of a specimen.

The predetermined biomarker is high-frequency microsatellite instability or a BRAF gene mutation, for example, but is not limited thereto. The predetermined biomarker may be low-frequency microsatellite instability, KRAS, SYNE1 (Spectrin Repeat Containing Nuclear Envelope Protein 1), A PC (antigen-presenting cells), TP53, TTN, or the like. The acquisition part 131 may acquire respective spatial distributions of a plurality of types of biomarkers. The feature value related to the predetermined biomarker may be a distribution of the biomarker itself, or may be a distribution of information indicating the degree of contribution to the estimation result of the biomarker expression level (for example, Attention Weight) estimated in a machine learning model that estimates the biomarker expression level from the input image data of a specimen for which the estimation is to be performed.

The predetermined protein is a CD3-positive lymphocyte or a CD20-positive lymphocyte, for example, but is not limited thereto. The predetermined protein may be a CD4-positive lymphocyte, a CD8-positive lymphocyte, Foxp3, PD-1, CD163Ave, CD155, or the like. The acquisition part 131 may acquire respective spatial distributions of the plurality of types of proteins.

The prognosis estimation part 132 inputs the input data including the spatial distribution acquired by the acquisition part 131 to the prognosis estimation model M1, and outputs the information output by the prognosis estimation model M1 as the estimated value of the prognosis of the target patient. With the information processing apparatus 1 configured in this manner, the spatial distribution of the biomarker or the predetermined protein in the specimen can be used for performing prognosis prediction of the target patient, and the accuracy of the disease prognosis prediction can be improved as compared with the existing prediction technique.

### [Biomarker Distribution Estimation]

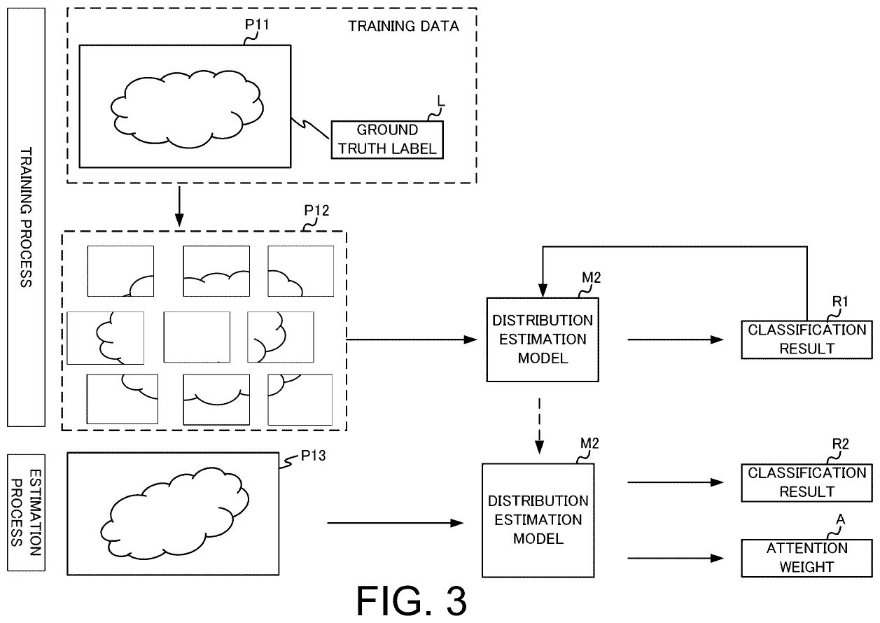

The information processing apparatus 1 may be configured to generate the spatial distribution of the feature value related to the predetermined biomarker in the specimen on the basis of image data obtained by imaging the specimen. FIG. 3 shows an example of processing in the distribution generation part 133 to estimate a spatial distribution of a feature value related to a biomarker. First, a training process for estimating a spatial distribution of a feature value related to a biomarker will be described. In the training process, the acquisition part 131 acquires image data P11 of the specimen and a ground truth label L assigned to the image data as training data. As one example, the training part 135 may use Multiple Instance Learning (MIL) in the training process. The ground truth label L is quantitative or qualitative information related to the biomarker in the entire specimen to be imaged. For instance, the ground truth label L is information indicating the degree of microsatellite instability for the entire specimen.

The acquisition part 131 divides the acquired image data P11 of the specimen into tiles. The training part 135 inputs a plurality of pieces of image data P12, which are obtained by dividing the image data P11, into the distribution estimation model M2 and outputs a classification result R1. The classification result R1 is information corresponding to the ground truth label L, and is a value estimated by the distribution estimation model M2 on the basis of the plurality of pieces of image data P12. The training part 135 feeds back a difference between the output classification result R1 and the ground truth label L to the distribution estimation model M2, and updates a parameter of the distribution estimation model M2. The training part 135 repeats the above process until a condition for ending the training is satisfied, and stores the trained distribution estimation model M2 in the storage 12. As a result, the storage 12 stores the distribution estimation model M2, which has been trained to output the spatial distribution of the feature value related to the predetermined biomarker in the image data upon input of the image data of the specimen.

Next, the inference processing will be described. The acquisition part 131 acquires image data P13 of the specimen collected from the target patient. The distribution generation part 133 inputs the image data of the specimen acquired by the acquisition part 131 into the distribution estimation model M2, and generates a spatial distribution. Specifically, the distribution generation part 133 divides the acquired image data P13 into tiles and inputs the tiles into the distribution estimation model M2. The distribution generation part 133 acquires the Attention Weight (A) when the distribution estimation model M2 estimates the classification result R2 of the image data P13. The Attention Weight (A) is a value indicating the degree to which each part of the image data contributes to classification when classifying the image data. Since the value of the Attention Weight (A) generated in this way indicates the contribution degree to the inference corresponding to the position in the image space, it can be used as the spatial distribution of the feature value related to the biomarker.

With the information processing apparatus 1 configured in this manner, it is possible to generate the spatial distribution of the feature value related to the predetermined biomarker in the specimen, thereby achieving the prognostic prediction with high accuracy using the spatial distribution of the feature value related to the predetermined biomarker as compared with the existing prediction technique.

### [Generating Spatial Distribution of Protein]

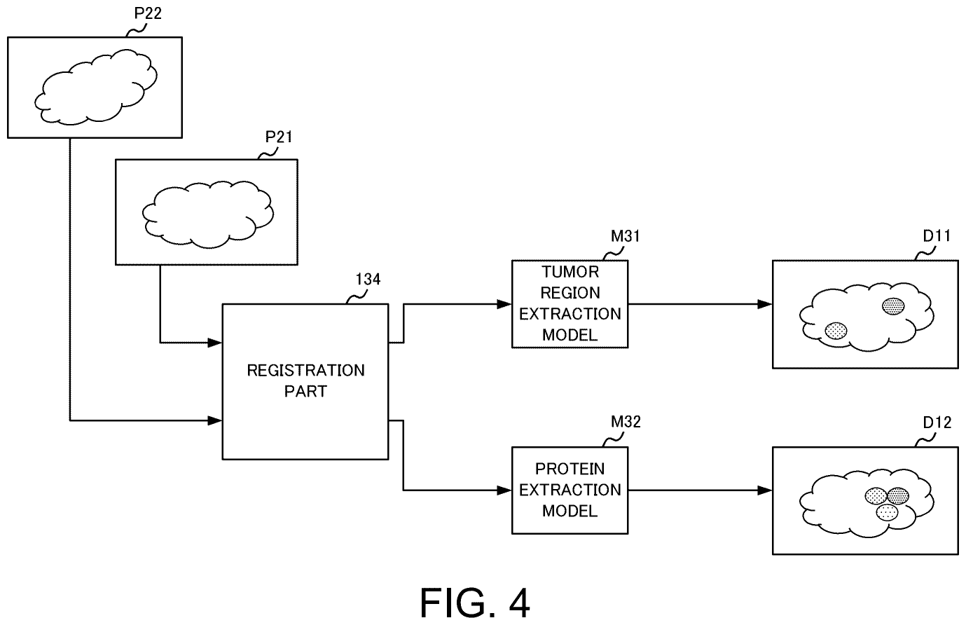

Next, a process for generating a spatial distribution of a predetermined protein will be described with reference to FIG. 4. The acquisition part 131 acquires first specimen image data P21 and second specimen image data P22 of the specimen collected from the target patient. The first specimen image data P21 is image data generated by i) processing the specimen collected from the target patient using a predetermined method (for example, staining) so as to enable detection of the cellular or tissue structure and ii) imaging the specimen. The second specimen image data P22 is image data obtained by i) processing the specimen collected from the target patient using a predetermined method so as to enable detection of the predetermined protein in the specimen and ii) imaging the specimen. The first specimen image data P21 and the second specimen image data P22, for example, are image data generated by i) slicing the collected specimen so that the cross-sections are parallel and have a uniform thickness, ii) staining the sliced specimen using the predetermined method, and iii) imaging the cross-sections of the specimen. The first specimen image data P21 and the second specimen image data P22 may be stained using different methods and imaged. As a method for staining the specimen, for example, a staining method for imaging the first specimen image data P21 is HE staining, and a staining method for imaging the second specimen image data P22 is IHC staining, but the method is not limited thereto. In the first specimen image data P21 and the second specimen image data P22, the cross-sections of the specimen that were in close proximity at the time of slicing are imaged. The first specimen image data P21 and the second specimen image data P22 may be data generated by imaging cross-sectional images after two regions of the specimen, which were not separated before the specimen was sliced, have been cut apart.

The registration part 134 registers corresponding positions of the image data with each other. The registration part 134 registers a position in the first specimen image data P21 and a position in the second specimen image data P22, acquired by the acquisition part 131, with each other. As an example, the registration part 134 registers the pieces of image data with each other by converting one of the pieces of image data so that a pixel in one piece of image data matches a corresponding pixel in the other piece of image data, using known deformable image registration.

The storage 12 stores a tumor region extraction model M31 and a protein extraction model M32. The tumor region extraction model M31 is a trained model that has been trained to output, upon input of the first specimen image data P21, a tumor region present in the specimen imaged in the image data. The training part 135 trains the tumor region extraction model M31 in advance using, as training data, the first specimen image data and the tumor region for training.

The protein extraction model M32 is a trained model that has been trained to output, upon input of the second specimen image data P22, a region in which predetermined protein is expressed in the specimen imaged in the image data. The training part 135 trains the protein extraction model M32 in advance using, as training data, the second specimen image data and the region in which the predetermined protein is expressed in the image data for training.

The distribution generation part 133 generates the spatial distribution of the tumor tissue and the predetermined protein in the specimen, on the basis of the first specimen image data P21 and the second specimen image data P22 acquired by the acquisition part 131. Specifically, the distribution generation part 133 inputs the first specimen image data P21 and the second specimen image data P22 acquired by the acquisition part 131 to the tumor region extraction model M31 and the protein extraction model M32, respectively, to output a tumor region D11 and a region D12 in which the predetermined protein is expressed. The tumor region D11 and the region D12 in which the predetermined protein is expressed, which are respectively output by the tumor region extraction model M31 and the protein extraction model M32, correspond to positions in the image data. Therefore, the tumor region D11 and the region D12 in which the predetermined protein is expressed respectively indicate the spatial distribution of the tumor tissue and the predetermined protein in the image data. The distribution generation part 133 outputs, to the acquisition part 131, the output spatial distributions of the tumor tissue and the predetermined protein.

The storage 12 stores the prognosis estimation model M1, which has been further trained using the spatial distributions of the tumor tissue and the predetermined protein in the specimen collected from the patient as an input. The acquisition part 131 acquires the spatial distributions of the tumor tissue and the predetermined protein in the specimen collected from the target patient. The acquisition part 131 may acquire the spatial distributions of the tumor tissue and the predetermined protein generated by the distribution generation part 133 on the basis of the first specimen image data and the second specimen image data generated by imaging the specimen of the target patient.

The prognosis estimation part 132 inputs, to the prognosis estimation model M1, the input data further including the spatial distribution of the tumor tissue acquired by the acquisition part 131 to output, as the estimated value of the prognosis of the target patient, the information output by the prognosis estimation model M1. With the information processing apparatus 1 configured in this manner, the prognosis prediction can be performed using information that associates the distribution of the tumor tissue with the distribution of the predetermined protein, enabling highly accurate prediction.

### [Processing in Information Processing Apparatus 1]

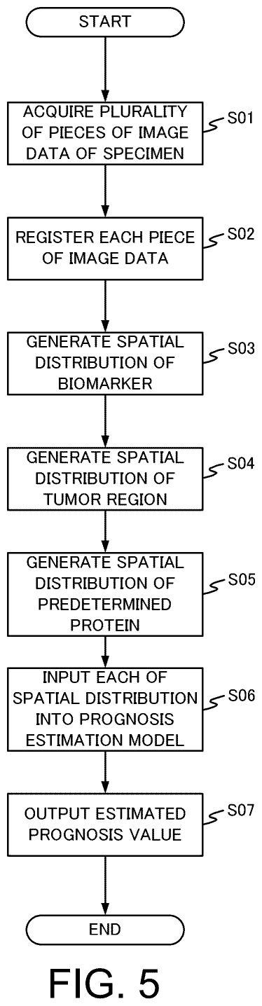

FIG. 5 is a flowchart illustrating processing in the information processing apparatus 1. The flowchart shown in FIG. 5 starts at a time point when an instruction to start an estimation process is received from an external device.

The acquisition part 131 acquires a plurality of pieces of image data of a specimen (S01). The registration part 134 registers each piece of the acquired image data (S02).

The distribution generation part 133 generates the spatial distribution of the feature value related to the predetermined biomarker on the basis of the acquired image data (S03). The distribution generation part 133 generates the spatial distribution of the tumor region on the basis of the acquired image data (S04). The distribution generation part 133 generates the spatial distribution of the predetermined protein on the basis of the acquired image data (S05).

The prognosis estimation part 132 inputs each of the spatial distributions generated by the distribution generation part 133 to the prognosis estimation model M1 (S06). The prognosis estimation part 132 outputs, as the estimated prognosis value, the estimated value output by the prognosis estimation model M1 (S07). Then, the information processing apparatus 1 ends the process.

### Modified Example 1

In the above description, an example of performing prognosis prediction of a patient having a predetermined disease has been described, but the information processing apparatus 1 may also be configured as an apparatus for estimating whether or not a predetermined drug is effective for a patient having a predetermined disease. In the following description, the same reference numerals are given to the same components as those already described, and redundant description is omitted.

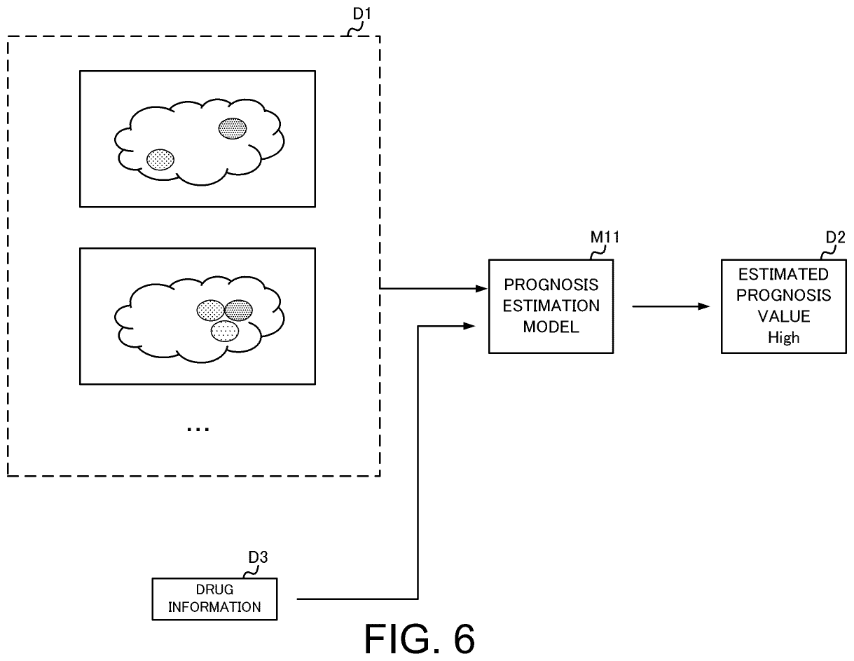

FIG. 6 is a diagram for explaining an overview of processing in the information processing apparatus 1 according to a first modified example. The information processing apparatus 1 according to the modified example differs from the information processing apparatus 1 shown in FIG. 1 in that the information processing apparatus 1 further acquires drug information D3 and inputs the acquired drug information into the prognosis estimation model M11 to output the estimated prognosis value D2 to the prognosis estimation model M.

Specifically, the acquisition part 131 further acquires information indicating a drug to be administered to the target patient. For example, the acquisition part 131 acquires the drug information D3 indicating a drug to be administered to the target patient from an external device which is not shown in figures. The drug information D3 may be information indicating a single type of drug or information indicating a plurality of types of drugs. Further, the drug information D3 may be information including i) a type of drug and ii) a dosage and an administration of a drug to be administered.

The storage 12 may store the prognosis estimation model M11, which has been further trained using information indicating a drug that has been administered to a patient as an input. That is, the prognosis estimation model M11 stored in the storage 12 in this case is a trained model that has been trained using, as training data, information including i) a spatial distribution of a feature value related to a predetermined biomarker in a specimen of a patient, ii) drug information indicating a drug that has been administered to the patient, and iii) prognosis information of the patient. When i) the spatial distribution of the feature value related to the predetermined biomarker and the like in the specimen collected from the target patient and ii) the drug information D3 indicating the drug that has been administered to the patient are input, the prognosis estimation model M11 stored in the storage 12 outputs the estimated prognosis value D2 indicating the prognosis of the patient.

The prognosis estimation part 132 further inputs the drug information D3 indicating the drug to be administered to the target patient acquired by the acquisition part 131 to the prognosis estimation model M11, and outputs, as the estimated prognosis value D2 of the target patient, the information output by the prognosis estimation model

M11. The prognosis estimation part 132 inputs, in addition to the spatial distribution, the information indicating the drug to be administered to the target patient acquired by the acquisition part 131 to the prognosis estimation model M11 stored in the storage 12, and outputs the estimated prognosis value D2 output from the prognosis estimation model M11.

### [Effects of the Information Processing Apparatus 1 According to Modified Example 1]

With the information processing apparatus 1 configured in this manner, the accuracy of estimating whether or not a drug is effective for a patient having a predetermined disease can be improved.

### Modified Example 2

It is known that the effectiveness of a drug varies due to the heterogeneity of a tumor tissue (the presence of various types of tissues). Accordingly, the information processing apparatus 1 is configured to further input the distribution of the feature value related to the tumor tissue and performs the prognosis prediction of the target patient, enabling performance of an estimation that takes into account the difference in prognosis due to the heterogeneity of the tumor tissue.

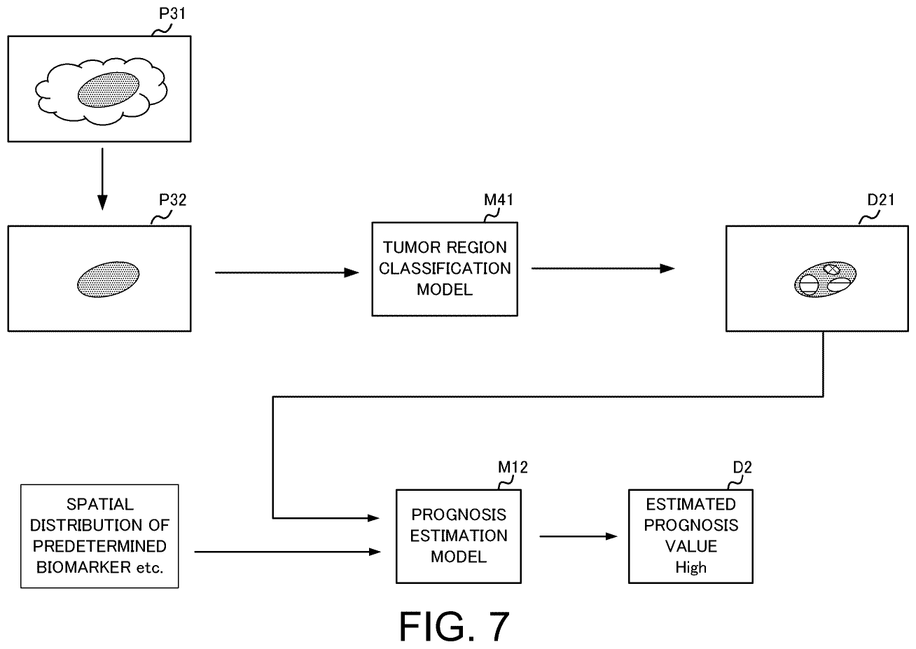

FIG. 7 is a diagram for explaining an overview of processing in the information processing apparatus 1 according to a second modified example. In this case, the storage 12 stores the prognosis estimation model M12, which has been further trained using the spatial distribution of the feature value related to the tumor tissue in the specimen collected from the patient as an input. The distribution generation part 133 generates tumor region image data P32 on the basis of first specimen image data P31 and the spatial distribution of the tumor region in the first specimen image data P31. The tumor region image data P32 is image data that includes information only about a region in which a tumor is present in the first specimen image data P31.

The distribution generation part 133 inputs the tumor region image data P32 into a tumor region classification model M 41, and outputs a feature value D21 for each region obtained by microscopically dividing the tumor region in the tumor region image data P32 (hereinafter referred to as a “patch”). The feature value D21 is a label indicating a cell density in a tumor cell or similar images on a patch-by-patch basis. The tumor region classification model M 41 is a trained model that has been trained by the training part 135 using tumor region image data as training data to output a feature value for each patch.

The acquisition part 131 acquires the spatial distribution of the feature value related to the tumor tissue in the specimen collected from the target patient. As one example, the acquisition part 131 acquires the feature value D21 of each patch obtained by microscopically dividing the tumor region generated by the distribution generation part 133 as the spatial distribution of the feature value related to the tumor tissue in the specimen collected from the target patient. The acquisition part 131 may acquire a spatial distribution of a feature value related to a tumor tissue in a specimen collected from a target patient from an external device.

The prognosis estimation part 132 outputs, as the estimated value of the prognosis of the target patient, information output by inputting the input data including the spatial distribution of the feature value related to the tumor tissue acquired by the acquisition part 131 to the prognosis estimation model M12. The prognosis estimation part 132 may further input, in addition to the spatial distribution of the feature value related to the tumor tissue, the spatial distribution of the feature value related to the predetermined biomarker and the like to the prognosis estimation model M12 to output the estimated prognosis value D2.

With the information processing apparatus 1 configured in this manner, it is possible to perform an estimation that takes into account a difference in prognosis due to the heterogeneity of the tumor tissue.

The present disclosure is explained on the basis of the exemplary embodiments. The technical scope of the present disclosure is not limited to the scope explained in the above embodiments and it is possible to make various changes and modifications within the scope of the disclosure. For example, all or part of the apparatus can be configured with any unit which is functionally or physically dispersed or integrated. Further, new exemplary embodiments generated by arbitrary combinations of them are included in the exemplary embodiments of the present disclosure. Further, effects of the new exemplary embodiments brought by the combinations also have the effects of the original exemplary embodiments.

What is claimed is:

1. An information processing apparatus comprising:
a storage that stores a prognosis estimation model, which has been trained to output a prognosis of a patient upon input of input data including a spatial distribution of at least one of a feature value related to a predetermined biomarker or a predetermined protein in a specimen collected from the patient;
an acquisition part that acquires a spatial distribution of at least one of a feature value related to a predetermined biomarker and a predetermined protein in a specimen collected from a target patient; and
a prognosis estimation part that outputs, as an estimated value of a prognosis of the target patient, information output by inputting input data including the spatial distribution acquired by the acquisition part to the prognosis estimation model.

2. The information processing apparatus according to claim 1, wherein
the storage stores the prognosis estimation model, which has been further trained using information indicating a drug that has been administered to a patient as an input,
the acquisition part further acquires information indicating a drug to be administered to the target patient, and
the prognosis estimation part further inputs, to the prognosis estimation model, a drug to be administered to the target patient acquired by the acquisition part to output, as the estimated value of the prognosis of the target patient, output information.

3. The information processing apparatus according to claim 1, wherein
the spatial distribution of the feature value related to the predetermined biomarker is a spatial distribution of a feature value related to high-frequency microsatellite instability or a BRAF gene mutation in the specimen collected from the patient.

4. The information processing apparatus according to claim 1, wherein
the storage further stores a distribution estimation model, which has been trained to output the spatial distribution of the feature value related to the predetermined biomarker in the image data upon input of image data of a specimen,
the acquisition part acquires image data of the specimen collected from the target patient, and
the information processing apparatus further includes a distribution generation part that generates the spatial distribution by inputting the image data of the specimen acquired by the acquisition part to the distribution estimation model.

5. The information processing apparatus according to claim 1, wherein
the spatial distribution of the predetermined protein is a spatial distribution of i) a tumor tissue and ii) a CD3-positive lymphocyte or a CD20-positive lymphocyte in the specimen collected from the patient.

6. The information processing apparatus according to claim 1, wherein
the storage stores the prognosis estimation model, which has been further trained using a spatial distribution of a tumor tissue and a predetermined protein in a specimen collected from a patient as an input,
the acquisition part further acquires a spatial distribution of a tumor tissue and a predetermined protein in a specimen collected from a target patient, and
the prognosis estimation part outputs, as the estimated value of the prognosis of the target patient, information output by inputting input data further including the spatial distribution of the tumor tissue and the predetermined protein acquired by the acquisition part to the prognosis estimation model.

7. The information processing apparatus according to claim 6, wherein
the acquisition part acquires i) first specimen image data, which is image data of the specimen collected from the target patient, the image data being obtained by imaging the specimen that has undergone predetermined processing to enable detection of a cellular or tissue structure in the specimen, and ii) second specimen image data, which is image data obtained by imaging the specimen that has undergone predetermined processing to enable detection of a predetermined protein in the specimen, and
the information processing apparatus further includes a distribution generation part that generates a spatial distribution of a tumor tissue and a predetermined protein in the specimen, on the basis of the first specimen image data and the second specimen image data acquired by the acquisition part.

8. The information processing apparatus according to claim 7, wherein
the first specimen image data and the second specimen image data are image data obtained by staining and imaging the specimen collected from the target patient using different methods, respectively,
the information processing apparatus further includes a registration part that registers a position in the first specimen image data with a position in the second specimen image data, and
the distribution generation part generates a spatial distribution of a tumor tissue and a predetermined protein in the specimen, on the basis of the first specimen image data and the second specimen image data registered with each other by the registration part.

9. The information processing apparatus according to claim 1, wherein
the storage stores the prognosis estimation model, which has been further trained using a spatial distribution of a feature value related to a tumor tissue in a specimen collected from a patient as an input,
the acquisition part further acquires a spatial distribution of a feature value related to a tumor tissue in a specimen collected from a target patient, and
the prognosis estimation part outputs, as the estimated value of the prognosis of the target patient, information output by inputting input data further including the spatial distribution of the feature value related to the tumor tissue acquired by the acquisition part to the prognosis estimation model.

10. The information processing apparatus according to claim 1, wherein
a prognosis estimation model is a trained model that has been trained using, as training data, i) a spatial distribution of a biomarker or protein in a specimen collected from a patient and ii) prognosis information indicating whether the patient survived for a predetermined period from a time point when the specimen was obtained.

11. An information processing method executed by a computer, the information processing method comprising the steps of:
acquiring a spatial distribution of at least one of a feature value related to a predetermined biomarker and a predetermined protein in a specimen collected from a target patient; and
outputting, as the estimated value of the prognosis of the target patient, information output by inputting, to a prognosis estimation model stored in a storage, input data including the spatial distribution acquired in the acquiring.

12. A non-transitory storage medium storing a program for causing a computer to realize steps of:
acquiring a spatial distribution of at least one of a feature value related to a predetermined biomarker and a predetermined protein in a specimen collected from a target patient; and
outputting, as the estimated value of the prognosis of the target patient, information output by inputting, to a prognosis estimation model stored in a storage, input data including the spatial distribution acquired in the acquiring.
